(19) United States
(12) Patent Application Publication
(10) Pub. No.: US 2008/0027539 A1
(43) Pub. Date: Jan. 31, 2008

(54) "W" Accommodating Intraocular Lens

(76) Inventor: J. Stuart Cumming, Laguna Beach, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 11/459,862

(22) Filed: Jul. 25, 2006

(51) Int. Cl. A61F 2/16

(52) U.S. Cl. 623/6.37

(57) ABSTRACT

An accommodating intraocular lens comprising a flexible body, a flexible optic which is moveable anteriorly and posteriorly relative to the lens body, and hinged portions longitudinally connecting the optic to the body. The body may have extending centration and fixation loops on its distal ends.

### BACKGROUND

Intraocular lenses have for many years had a design of a single optic with loops attached to the optic to center the lens and fixate it in the empty capsular bag of the human eye. In the mid '80s plate lenses were introduced, which comprised a silicone lens, 10.5 mm in length, with a 6 mm optic. These lenses could be folded but did not fixate well in the capsular bag, but resided in pockets between the anterior and posterior capsules. The first foldable lenses were all made of silicone. In the mid 1990s an acrylic material was introduced as the optic of lenses. The acrylic lens comprised a biconvex optic with a straight edge into which were inserted loops to center the lens in the eye and fixate it within the capsular bag.

Recently accommodating intraocular lenses have been introduced to the market, which generally are modified plate haptic lenses. A plate haptic lens may be defined as an intraocular lens having two or more plate haptics where combined junctions with the optic represent one quarter or more of the circumference of the optic.

Flexible acrylic material has gained significant popularity among ophthalmic surgeons. In 2003 for example more than 50% of the intraocular lenses implanted had acrylic optics. Hydrogel lenses have also been introduced. Both the acrylic and hydrogel materials are incapable of multiple flexions without fracturing.

The advent of an accommodating lens which functions by moving the optic along the axis of the eye by repeated flexions somewhat limited the materials from which the lens could be made. Silicone is the ideal material, since it is flexible and can be bent probably several million times without showing any damage. Additionally one or more grooves or hinges can be placed across the plate adjacent to the optic as part of the lens design to facilitate movement of the optic relative to the outer ends of the haptics. An example accommodating lens of this nature is disclosed in U.S. Pat. No. 6,387,126 in the name of J. Stuart Cumming.

### SUMMARY OF THE INVENTION

According to the present invention a new form of accommodating intraocular lens having a lens body and optic is provided with plural straps, preferably two, between the optic and lens body to allow the optic to move anteriorly and posteriorly in response to the pressure gradient created with accommodation. The lens body preferably has a central hinge. The structure is such that it particularly benefits from changes in vitreous pressure with accommodation.

Thus, it is a feature of the present invention to provide a new form of accommodating lens.

### BRIEF DESCRIPTION OF THE DRAWINGS

FIG. 1 is a prospective view of the front or anterior side of the lens according to the present invention.

FIG. 2 is a plan view of the anterior side.

FIG. 3 is a plan view of the back or posterior side of the lens.

FIG. 4 is a side view.

FIG. 5 is an end view.

FIG. 6 is a cross-sectional view along lines 6-6 of FIG. 2.

FIG. 7 is a perspective view of the back or posterior side of the lens.

FIG. 8 is a side view during distance vision.

FIG. 9 is a side view during near vision.

### DESCRIPTION OF PREFERRED EMBODIMENT

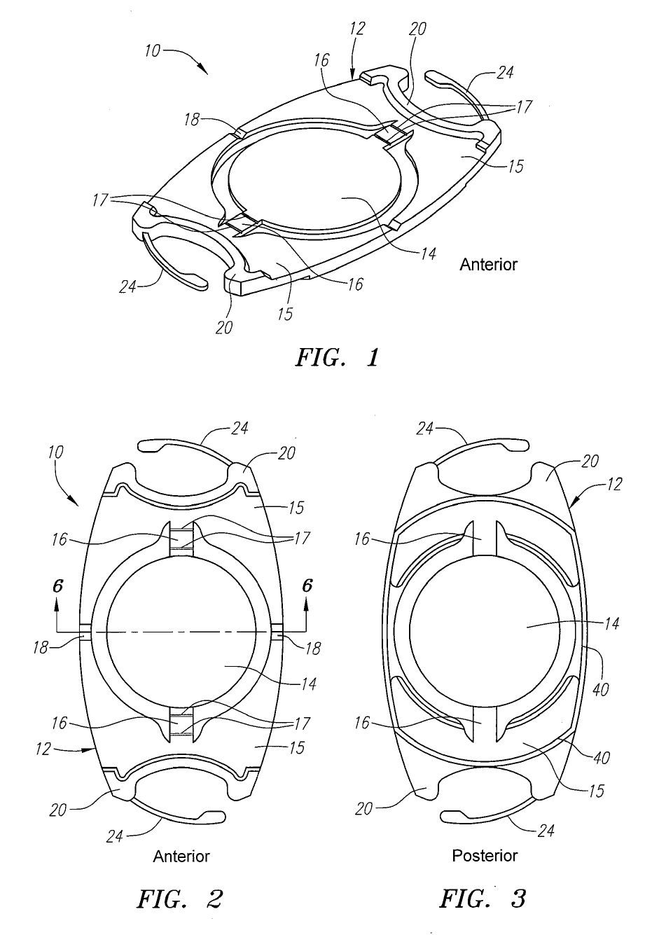

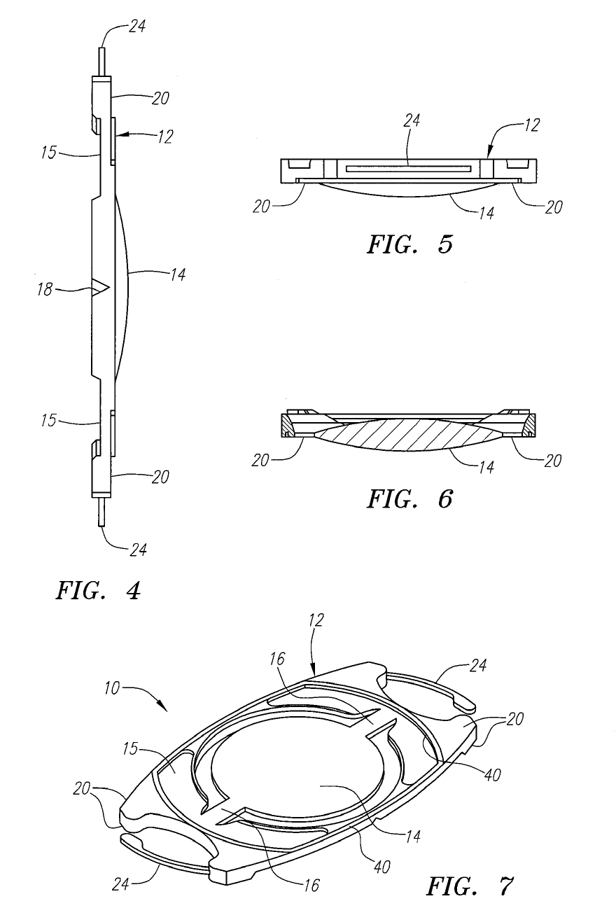

Turning now to the drawings, FIG. 1 is a perspective view of the present lens 10 including a lens body or plate 12 and optic 14. The body 12 includes haptics 15. The body 12 and optic 14 are formed of silicone or other suitable flexible material. Straps 16 are provided between the body 12 and the periphery or outer diameter of the optic 14. Each strap preferably includes one or two hinges 17 on the anterior side of the lens, or no hinges. The straps may be 0.5 mm long in the radial direction and 0.25 mm thick to support the optic 14 by the straps 16. The optic 14 typically can have a diameter of 4.5-5.0 mm, a typical width of the overall lens 10 on the short side is 6.1 mm and the typical length from end to end (not including fixation fingers) on the long side is 10.5-11.5 mm. A typical optic thickness is 0.4-1.3 mm.

The body 12 and optic 14, as well as outer thickened footplate ends 20, are formed of silicone or other suitable flexible material. The lens 10 also preferably includes fixation loops 24 of polymide or similar material. A typical outer loop-to-loop length is 11.0-12.5 mm. The thickened ends 20 fully engulf the fixation loops 24 in the silicon thus to provide a strong matrix to hold the loops 24. There is an additional function of these thickened areas of the plate. They also serve to elevate the anterior capsule of the human lens away from the optic and from the posterior capsule after the cataract has been removed and the lens implanted. This may serve to reduce capsular pacification and contraction. The haptics 15 can be any typical shape, such as in the present Figures, rectangular, triangular, or the like.

The straps 16 and hinges 17 function by allowing the optic to move anteriorly and posteriorly. The approximately 1.0-2.0 mm wide straps are a point of relative weakness in the plane of the lens body 12 encircling the optic 14, thereby allowing the entire optic 14 to herniate forward (anteriorly) from its far posterior position in a translational forward movement. This feature is enhanced by keeping the mass of the optic 14 to a minimum as described below. This new mechanism may boost the effect of the other features of the lens. Rather than a fluid-filled sac pushing through an aperture as in some prior lenses, the present lens involves a deformable solid optic moving anteriorly and posteriorly through a hinged area 16 in the plate or body 12. Hinges 18 on the anterior side of the body 12 hinging the haptics 15 further facilitate movement of the optic with ciliary muscle contraction.

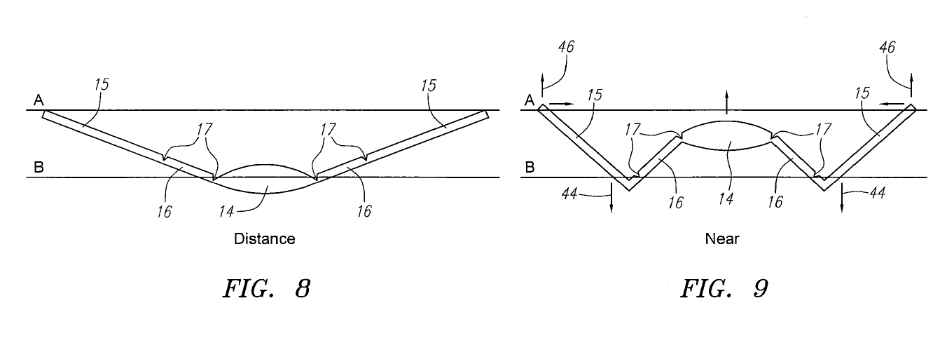

Of significance is the manner in which the optic 14 and haptic plates 15 move in accommodating from distance to near vision and this is particularly illustrated in FIGS. 8 and 9 with respect to anterior A and posterior B reference lines in these Figs. As is usual, the ends of the haptics 15 or the loops 24 as the case may be are implanted in the capsular bag such that the optic 14 is vaulted posteriorly for distance vision as seen in FIG. 8. The optic 14 moves anteriorly for near vision as shown in FIG. 9 upon ciliar muscle contraction. In particular, the whole lens moves forward a little as seen in FIG. 9 and the haptic plates 15 move centrally and backward slightly in the direction of arrows 44 which results in a pressure caused by ciliary muscle contraction in further increase in pressure on the optic 14 that pushes the optic further forward because the hinge or hinges 17 are thin and stretch a little and the optic deforms somewhat. This provides an increase in anterior optic movement with optic deformation. Also, the ends of the haptics 15 push backward against the posterior capsule as indicated by arrows 46 in FIG. 9 with increases of vitreous pressure.

The width of the hinges is 1.0-3.0 mm and the thickness of 0.1-0.3 mm.

Another feature allowing the present lens to accommodate is that the optic 14 can be deformable and constructed with a lower durometer than previously built into any lens. The surrounding plate 12 preferably is made of a higher, standard durometer material, similar to the eyeonics Inc. AT45 lens (which is durometer 48). The optic 14 itself is not required to contribute to the structural stability of the lens and, therefore, the optic 14 can be extremely soft. In addition to forward axial translation, the bending or deformation of the optic 14 with accommodation will induce power change. This may result in the bending of the optic to be accentuated. This feature is further enhanced by maintaining the optic very thin since a thinner optic will bend more than a thick optic for any given level of force applied. An example range of optic 14 center thicknesses is about 0.4 mm to 1.3 mm for a diopter range of 10 to 33. A typical common diopter of the optic of the present lens is 22 diopters and which has a thickness of 0.73 mm. As a comparison, the AT 45 noted earlier in a 22 diopter has a thickness of 0.88 mm, and a newer AT-45SE is 0.98 mm.

A 4.5 mm diameter optic 14 and with a reduced edge thickness of 0.1 to 0.2 mm for example can be provided. The index of refraction can be increased and this will accentuate this feature even further. Optic flexure is a new and poorly understood phenomenon, and unwanted optical distortion may be encountered resulting in poor vision either at near or far distances, in which case the durometer of the material will need to be raised.

The present lens can be easily foldable with forceps or an injector. A pre-loaded system is preferable.

An additional feature is the incorporation of a ridge or ridges 40 on the back surface (posterior side) of the plate 12 and/or haptic arm as the case may be as seen in FIGS. 3 and 7. These ridges traverse the plate and completely encircle the optic around the perimeter of the lens body. There is an additional ridge central to the first ridge traversing the plate adjacent to the optic straps. The purpose of these ridges is to prevent proliferation of lens epithelial cells into the area behind the plate or optic. For plate lenses this can dramatically reduce the incidence of capsular contraction. Epithelial cells will be prevented from migrating under the plate and undergoing a fibrotic contraction. Furthermore, the square edge of the loops, plate haptics and the square edge of the optic further protect against cells migrating in from the sides of the plate.

While an embodiment of the present invention as been shown and described, various modifications may be made without departing from the scope of the present invention, and all such modifications and equivalents are intended to be covered.

What is claimed is:

1. An accommodating intraocular lens comprising a flexible body such that upon constriction of the ciliary muscle a proximal plate thereof moves posteriorly, and a flexible optic surrounded by the body and mounted to the body by hinged straps to move anteriorly and posteriorly upon ciliary muscle relaxation relative to the lens body.

2. A lens as in claim 1 wherein the body has at least one hinge across the body.

3. A lens as in claim 2 wherein the hinge is across the middle of the body.

4. A lens as in claim 1 wherein each strap is on opposite sides of the optic longitudinal to the lens body and each strap has one hinge.

5. A lens as in claim 1 wherein each strap is on opposite sides of the optic longitudinal to the lends body and each strap has two hinges.

6. A lens as in claim 5 wherein the body has a central hinge on the anterior side of the body.

7. A lens as in claim 1 further including loops extending from outer ends of the body.

8. A lens as in claim 1 wherein a posterior side of the body includes a ridge.

9. An accommodating intraocular lens comprising a flexible body and a flexible optic, the flexible optic being mounted longitudinally to the body by thin flexible straps disposed on each side of the optic between the optic and the body, the straps being approximately 0.5 mm long radially and approximately 0.25 mm thick, and the lens including a plurality of fixation loops attached to ends of the body.

10. A lens as in claim 9 including haptics with a weakened thinned or hinged portion within the lens body allowing the lens optic, its surrounding lens body part central to the hinge, to move forward and backward relative to the outer ends of the haptics.

11. A lens as in claim 10 wherein the hinge is in the middle of the body on the anterior side.

12. A lens as in claim 9 wherein the outer ends of the haptics include loops to securely fixate and center the lens.

13. A lens as in claim 9 wherein the optic is constructed to optically deform with ciliary muscle contraction and vitreous pressure to enhance near vision.

14. A lens as in claim 9 wherein the lens body has a central hinge on the anterior side.
